(19) United States
(12) Patent Application Publication
(10) Pub. No.: US 2006/0004294 A1
(43) Pub. Date: Jan. 5, 2006

(54) Method and heart-rate monitor

(75) Inventors: Jouni Juntunen, Helsinki (FI); Panu Kekalainen, Espoo (FI); Kauko Pienimaki, Helsinki (FI); Kalle Mannonen, Kirkkonummi (FI)

Correspondence Address: BIRCH STEWART KOLASCH & BIRCH, PO BOX 747, FALLS CHURCH, VA, 22040-0747, US

(73) Assignee: SUUNTO OY

(21) Appl. No.: 11/169,712

(22) Filed: Jun. 30, 2005

Related U.S. Application Data
(60) Provisional application No. 60/585,225, filed on Jul. 2, 2004.

(51) Int. Cl. A61B 5/02

(52) U.S. Cl. 600/500; 128/903

(57) ABSTRACT

The invention relates to a method and a heart-rate monitor. The invention is based on heartbeats being detected in a remote device and a time datum relating to each heartbeat being recorded. The time datum is expressed in some mutually comparably form, for example, as a value given by the internal clock of the measuring device, or a similar device expressing the passage of time. After this, the time data relating to heartbeats are sent to a heart-rate monitor, wristop computer, or similar terminal device, together with an identifier datum. The pulse data and, if necessary, pulse-interval data can be calculated in the terminal device and pulse data that may be lacking can be detected on the basis of the time data obtained.

The present invention relates to a method, according to the preamble of Claim 1, for transmitting heart-rate data from a remote device to a terminal device.

The invention also relates to a method, according to the preamble of Claim 15, for processing the received heart-rate data in the terminal device.

The invention also relates to heart-rate monitors and the transmitter parts and calculation and display units of heart-rate monitors, which are arranged to use the aforementioned methods. The term heart-rate monitor thus also refers to a wristop computer or some other corresponding device, equipped with heart-rate monitor properties.

According to the prior art, heart-rate monitors and similar wristop computers typically include a transmitter belt attached to the human body by a flexible belt, which nowadays typically measures the pulse. This measuring device equipped with electrodes transmits a measuring message by radio to a wristwatch-like wristop computer, in which at least part of the received signal is processed and displayed on the display of the wristop computer. Known wristop computers can be used to measure not only pulse, but also, for example, blood pressure, speed, acceleration, distance travelled, and direction data.

If there several sportspeople using wristop computers are close to each other, which typically happens in exercise sessions with instructors, or in mass training events, the receiver must identify the correct transmitter.

According to the prior art, two alternative methods are used to identify the correct transmitter. For instance, Finnish patent 96380 discloses an analog solution, in which the time between at least two identifier pulses is used as the identifier data, by means of which the receiver can select the correct transmission signal from a group of several transmission signals. Implemented using analog technology, this system has been known to lead to error states, if the distance between the identifier pulses of two devices has been too close to each other. This has permitted, for example, due to interference, the signals of two transmitters to be mistaken for each other, at least at times.

On the other hand, in newer devices, digital signal transfer with digital identification codes has been used. One such digital solution is disclosed in patent publication EP 0 760 224 B1. In this solution, the transmitter belt includes a set of circuits, which are used to detect the heartbeats and create pulse-interval information, which states the length of time between the detected heartbeats. The pulse-interval data is coded into a digital signal, which is transmitted to the wristop computer.

Though it is certainly possible, on the basis of the known digital solution, to solve the aforementioned problem of the mixing of the transmitted and received information, we have observed that, in the known digital solution itself, problems may arise in a situation in which heartbeats are not detected, or the pulse-interval information cannot, for some reason, be transmitted to the wristop computer. It may be very difficult for the wristop computer to manage a situation, wherein pulse-interval information is missing. This lack of pulse-interval information may lead to confusion in the wristop computer and to an error in the displayed pulse information.

The invention is intended to create a new type of solution, in which there is a relatively small possibility of confusing the data transmitted between different wristop-computer/transmitter-belt pairs, and of internal confusion in a wristop-computer/transmitter-belt pair.

The invention is based on the fact that, in the transmitter belt or similar measuring device (the remote device), the heartbeats are detected and the time data relating to each heartbeat is stored (temporarily). The time data is expressed in some mutually comparable form, for example, as a value given by the internal clock of the measuring device, or some similar device expressing the passage of time. The time data can correspond to the local time of day, or be entirely independent of it. The time data should depict, with appropriate precision and mutually comparably, the moments in time at which the consecutive pulses occur. After this, the time data relating to the heartbeats are transmitted to the heart-rate monitor, wristop computer, or similar terminal device, along with identifier data. The pulse data and, if necessary, the pulse-interval data can now be calculated in the terminal device and pulse data that may be missing can be detected on the basis of the time data obtained.

More specifically, the transmission method according to the invention is characterized by what is stated in the characterizing portion of Claim 1.

The processing method according to the invention is, in turn, characterized by what is stated in the characterizing portion of Claim 15.

For their part, the characteristic features of the transmission components and calculation and display units of the heart-rate monitor according to the invention are defined in Claims 14 and 26.

Considerable advantages are gained with the aid of the invention. This is because, with the aid of the invention, it is possible to achieve a solution, in which there is relatively little possibility of confusing the data transmitted between different terminal-device/remote-device pairs, (for example, a wristop-computer/transmitter-belt pair), and of internal confusion in a terminal-device/remote-device pair.

Compared to the known digital solution, the advantages are achieved of both the easy transmission of sufficient data from a remote device (for example, a transmitter belt) to a terminal device (for example, a wristop computer) for it to be possible to calculate pulse data and also of being able to determine if the pulse data received is deficient.

An embodiment using a digital identifier gains the advantage over the analog coding technique of being able to use a considerably larger number of transmitter-receiver combinations close to each other, without uncertainty arising as to the receiver for which each signal is intended.

The embodiments of the invention permit the provision of very accurate pulse data and pulse-interval data to a wristop device or computer. Accurate pulse-interval data can be exploited, for example, when calculating variations of the pulse intervals, in a wristop device or computer, in post-exercise analysis. Pulse-interval data or variation in the pulse intervals can be used to obtain interesting information, for example, on the level of stress of the body. In this respect too, the embodiments of the invention differ substantially from the technique disclosed in publication EP 0 760 224 B1, with the aid of which in practice it is impossible to achieve an accuracy sufficient to allow the utilization of pulse-interval data in a more highly-developed analysis of the pulse data.

In the following, the invention is examined with the aid of application examples according to the accompanying drawings.

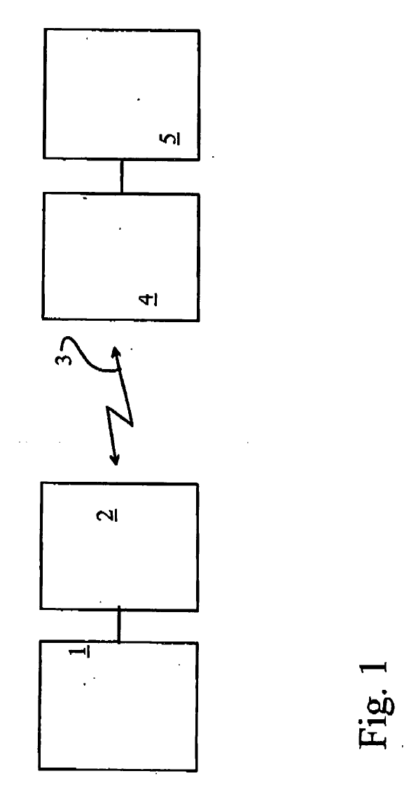

FIG. 1 shows a block diagram of one device environment, in which the invention can be exploited.

According to FIG. 1, the apparatus includes a measuring device 1, which is typically a pulse meter attached to the chest by a flexible belt. The pulse meter in question contains electrodes, with the aid of which the pulse of the person is measured. The measuring device 1 can naturally be some other measuring device. The measuring device is connected to a transmitter/coder 2, in which the measurement signal is edited into a transmittable form and given a code individuating the transmitter 2. The signal is sent from the transmitter 2 wirelessly over a transfer path 3 to a receiver 4, which also includes means for decoding the code. The transfer path 3 is typically the air space between the measuring device 2 located around the chest and a receiver 4 located on the wrist.

The receiver 4 is, in turn, connected to a data-processing unit 5, to which a display is typically also connected. The receiver 4 and the data-processing unit 5 are typically implemented in a wristop computer, which is reminiscent of a wristwatch. Such a wristop computer can include not only pulse-measuring properties or other measuring properties, but also normal clock functions, possibly positioning equipment, such as GPS circuits, and an altimeter, in which the sensor is typically a pressure sensor.

The wristop computer can also include, for example, a temperature measuring device. Also known are wristop computers with connections and data communications devices for connecting the wristop computer to a normal microcomputer, for example, through a USB bus.

In the following, methods according to the embodiments are described, for transmitting pulse data from a remote device to a terminal device. In such methods:

a message, which includes both pulse data and an identifier, is produced in the remote device, and

the message is transmitted from the remote device to the terminal device.

A characteristic feature of the embodiments is that the pulse data includes time data relating to the heartbeats.

The identifier used in the embodiments can be in an analog form, or preferably in a digital form.

In one embodiment, the pulse data is coded into a digital form in the remote device, before transmission. In a second embodiment, the information contained in the message being transmitted to the terminal device is entirely coded into a digital form.

In the remote device, data from several heartbeats can also be collected and the time data relating to the several heartbeats can be incorporated in a single message. The pulse data contained in each message can include, for example, a predefined number of time data, which relate to detected consecutive heartbeats. The number of time data contained in the message can be, for example, from 1 to 8, preferably from 2 to 4, and most preferably exactly 3.

The message containing pulse data is produced in the remote device, without calculating the pulse-interval data. Thus the remote device does not necessarily need a functionality of this kind at all.

The identifier in a digital form contained in the message individuates the remote device, allowing the terminal device to identify the remote device with the aid of the digital-form identifier.

Further, according to an embodiment, the time data includes a value obtained from a time reference, which is selected on the basis of the moment of detecting the heartbeat. This typically takes place by the time value being retrieved from the time reference immediately after the detection of the heartbeat. The time value is thus received after an operating delay of the device from the detection of the heartbeat. As the device's operating delay remains essentially constant, the time values obtained will be mutually comparable, as the same operating delay relates to each time value. The time reference used is typically an internal clock device in the remote device.

Thus the values obtained from the time reference can be relative, as they express the moment of the heartbeat relative to the moment of the preceding or following heartbeats. Alternatively, the values obtained from the time references can be essentially absolute, by expressing with substantial accuracy the heartbeat moment in local time.

It should be further stated that, in the most usual embodiment, the messages are sent wirelessly using a radio frequency.

The aforementioned operations can thus be performed in a remote device, for example, in the transmitter part of a pulse meter. In the terminal device, for example, in the calculation and display unit of the pulse meter, the operations depicted in the following can, for their part, be performed.

According to the embodiments, the terminal device receives messages containing pulse data and an identifier, sent by the remote device and defines the current pulse value on the basis of the pulse data contained in the received messages. In the embodiments, the pulse data comprises time data relating to the heartbeats.

The identifier used in the embodiments can be in an analog form, or preferably in a digital form.

In one embodiment, the pulse data received is coded into a digital form. In a second embodiment, the information contained in the received message is entirely coded into a digital form.

The received message can include time data relating to one or more heartbeats. In some embodiments, the pulse data contained in each message comprises a predefined number of time data, which relate to consecutive heartbeats detected in the remote device. The number of time data contained in the message can be, for example, from 1 to 8, preferably from 2 to 4, and most preferably exactly 3.

As, in this method, the pulse-interval information need not be calculated in the remote device, the pulse-interval data is calculated in the terminal device on the basis of the received time data, if information is required on the time that has elapsed between the heartbeats.

To prevent confusion in the terminal device between different pairs of terminal-devices/remote-devices, it is advantageous to examine the digital-form identifier contained in each message and, if the identifier differs from the identifier set in the terminal device, to reject a message containing a differing identifier.

Thus, each time datum can include a relative time value, so that the time values express the moment of the heart beating, relative to the moment of the preceding or following heartbeats, or an essentially absolute time value, so that the time values express, with substantial accuracy, the moment of the heartbeat in local time.

The message is typically received wirelessly using a radio frequency.

1. A method for transmitting pulse data from a remote device to a terminal device, the method comprising:
in the remote device, producing a message, which contains pulse data and an identifier, and
transmitting the message from the remote device to the terminal device,
characterized in that the pulse data comprises time data relating to a heartbeat.

2. A method according to claim 1, characterized in that the pulse data is coded into a digital form in the remote device, before transmission.

3. A method according to claim 1 or 2, characterized in that the information contained in the message transmitted from the remote device to the terminal device is entirely coded into digital form.

4. A method according to any of claims 1-3, characterized in that data on several heartbeats is collected in the remote device and time data relating to the several heartbeats is included in a single message.

5. A method according to any of claims 1-4, characterized in that the pulse data of each message comprises a predefined number of time data, which relate to the detected consecutive heartbeats.

6. A method according to claim 4 or 5, characterized in that the number of time data contained in the message is from 1 to 8, preferably from 2 to 4, and most preferably exactly 3.

7. A method according to any of claims 1-6, characterized in that the message containing pulse data is produced in the remote device without calculating the pulse-interval data.

8. A method according to any of claims 1-7, characterized in that the identifier is in a digital form and individuates the remote device, so that the terminal device can identify the remote device with the aid of the identifier in a digital form.

9. A method according to any of claims 1-8, characterized in that the time data contain a value obtained from a time reference and selected on the basis of the moment of the detection of the heartbeat.

10. A method according to claim 9, characterized in that an internal clock device of the remote device is used as the time reference.

11. A method according to claim 9 or 10, characterized in that the values obtained from the time reference are relative, in such a way that they express the moment of a heartbeat relative to the preceding or following heartbeats.

12. A method according to claim 9 or 10, characterized in that the values obtained from the time reference are essentially absolute, in such a way that they express with substantial accuracy the moment of a heartbeat in local time.

13. A method according to any of claims 1-12, characterized in that the message is transmitted wirelessly using a radio frequency.

14. A transmitter part of a heart-rate monitor, for transmitting to a calculation and display unit, characterized in that the transmitter part is arranged to implement the method according to any of claims 1-13.

15. A method for processing in a terminal device pulse data received from a remote device, the method comprising:
receiving messages, which contain pulse data and an identifier, transmitted by the remote device, and
determining the pulse value at the moment on the basis of the pulse data contained in the received messages,
characterized in that the pulse data comprises time data relating to a heartbeat.

16. A method according to claim 15, characterized in that the received pulse data is coded into a digital form.

17. A method according to claim 15 or 16, characterized in that the information contained in the received messages is entirely coded into a digital form.

18. A method according to any of claims 15-17, characterized in that the received message contains time data relating to several heartbeats.

19. A method according to any of claims 15-18, characterized in that the pulse data contained in each message comprises a predefined number of time data, which relate to consecutive heartbeats detected in the remote device.

20. A method according to claim 18 or 19, characterized in that the number of time data contained in a message is from 1 to 8, preferably from 2 to 4, and most preferably exactly 3.

21. A method according to any of claims 15-20, characterized in that, if data on the duration of the time between heartbeats is required, the pulse-interval data is calculated on the basis of the time data received.

22. A method according to any of claims 15-21, characterized in that the identifier contained in each message is examined, and, if the identifier differs from the identifier set for the terminal device, the message containing the differing identifier is rejected.

23. A method according to any of claims 15-22, characterized in that each time data contains a relative time value, in such a way that the time values express the moment of the heartbeat relative to the moments of the preceding or following heartbeats.

24. A method according to any of claims 15-22, characterized in that each time datum contains an essentially absolute time value, in such a way that the time values express with substantial accuracy the moment of a heartbeat in local time.

25. A method according to any of claims 15-24, characterized in that the message is received wirelessly using a radio frequency.

26. A calculation and display unit of a heart-rate monitor, characterized in that it is arranged to implement the method according to any of claims 15-25.
